(19) United States
(12) Patent Application Publication
(10) Pub. No.: US 2026/0272860 A1
(43) Pub. Date: Sep. 17, 2026

(54) COMPOSITIONS FOR THE CONTROLLED-RELEASE OF CHOLINE

(71) Applicant: BALCHEM CORPORATION, New Hampton, NY (US)

(72) Inventors: Abnor Ibraimi, New Hampton, NY (US); Andrea Montebelli, Bertinoro (IT); Jerome Lynn Freiburger, Verona, MO (US); Cecilia Sbrighi, Bertinoro (IT)

(21) Appl. No.: 19/078,536

(22) Filed: Mar. 13, 2025

(51) Int. Cl. A61K 31/14; A61K 9/50

(52) U.S. Cl. CPC A61K 31/14; A61K 9/5015

(57) ABSTRACT

Coated microgranules are descried and include an extruded core and substantially wax free coating. The extruded core is substantially wax-free and contains a choline salt and a hydrogenated seed oil or a hydrogenated vegetable oil. The substantially wax free coating contains a hydrogenated seed oil, palm oil, or a combination thereof. Feedstuffs containing the coated microgranules and methods of delivering choline to the lower digestive tract to a ruminant animal by administering the feedstuffs to the ruminant animal are also provided.

### TECHNICAL FIELD

The present disclosure relates to compositions for the controlled-release of choline.

### BACKGROUND

Supplementing farm animal feedstocks is a common practice to improve health conditions, increase productive longevity and increase zootechnical performance. In order for this to occur, the active nutrients must substantially be absorbed in the intestines and abomasum of the farm animal.

However, since feedstocks are administered by mouth to the animals, the nutrients are often enzymatically, chemically degraded in the first section of the animal digestive tract, i.e., the rumen, before reaching the intestines or abomasum. Thus, farm animals do not absorb the nutrients provided by the feedstocks.

What is needed are farm animal feedstocks that increase the post-rumen absorbance of nutrients in farm animal feedstocks.

### SUMMARY

In some embodiments, the disclosure provides coated microgranules. The coated microgranules comprise an extruded core that is substantially wax-free and comprises about 55 to about 65 wt. % of the coated microgranule, of a choline salt; about 2.8 to about 3.8 wt. % of the coated microgranule, of silicon dioxide; about 1.4 to about 2 wt. % of the coated microgranule, of calcium stearate; about 4 to about 5 wt. % of the coated microgranule, of a hydrogenated seed oil or a hydrogenated vegetable oil; and about 1.1 to about 2.1 wt. % of the coated microgranule, of lecithin. The coated microgranules also comprise a substantially wax free coating comprising about 26 to about 32 wt. % of the coated granule of a hydrogenated seed oil, palm oil, or a combination thereof.

In other embodiments, the disclosure provides feedstuffs comprising the coated microgranule disclosed herein.

In further embodiments, the disclosure provides methods of delivering choline to the lower digestive tract to a ruminant animal, comprising administering the feedstuff disclosed herein to the ruminant animal.

### BRIEF DESCRIPTION OF THE DRAWINGS

FIG. 1 is a bar graph for the microgranules of Example 1 compared to the prior art microgranules showing the bypass, digestibility and bioavailability. In this figure, the first bar is each set is the prior art microgranules and the second bar in each set is the Example 1 microgranules.

FIG. 2 is box plot for digestibility. In this figure, the left bar is the prior art microgranules and right bar is the Example 1 microgranules.

FIG. 3 is box plot for bioavailability. In this figure, the left bar is the prior art microgranules and right bar is the Example 1 microgranules.

### DETAILED DESCRIPTION OF ILLUSTRATIVE EMBODIMENTS

The disclosure may be more fully appreciated by reference to the following description, including the following glossary of terms and the concluding examples. It is to be appreciated that certain features of the disclosed compositions and methods which are, for clarity, described herein in the context of separate aspects, may also be provided in combination in a single aspect.

In the following descriptions of exemplary embodiments of the present invention, all references, including publications, patent applications, and patents, cited herein are incorporated by reference into this application to the same extent as if each reference were individually and specifically indicated to be incorporated by reference and were set forth in its entirety herein.

Conversely, various features of the disclosed compositions and methods that are, for brevity, described in the context of a single aspect, may also be provided separately or in any subcombination.

In the disclosure, the singular forms “a,” “an” and “the” include the plural reference, and reference to a particular numerical value includes at least that particular value, unless the context indicates otherwise. For example, reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

When a list is presented, unless stated otherwise, it is to be understood that each individual element and every combination is to be interpreted as separate embodiments. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself.

It is noted that the claims may be drafted to exclude an optional element. As such, this statement is intended to serve as antecedent basis for use of such exclusive terminology as “only” and the like in connection with the recitation of claim elements, or use of a “negative” limitation.

### Microgranules

The disclosure provides coated microgranules that provide good bioavailability of nutrients to farm animals. The term “microgranules” as used herein refers to solid particles having a cylindrical shape. One of skill in the art would be able to select a suitable microgranule size, e.g., by selection of the extruder die. According to the disclosure, the microgranules have a width diameter of about 1.2 to about 2.2 mm. In some embodiments, the microgranules have a width diameter of about 1.2, about 1.3, about 1.4, about 1.5, about 1.6, about 1.7, about 1.8, about 1.9, about 2, about 2.1, or about 2.2 mm. In other embodiments, the microgranules have a width diameter of about 1.2 to about 2, about 1.2 to about 1.8, about 1.2 to about 1.6, about 1.4 to about 2.2, about 1.4 to about 2, about 1.4 to about 1.8, about 1.4 to about 1.6 m about 1.6 to about 2.2, about 1.6 to about 2, about 1.6 to about 1.8, about 1.8 to about 2.2, about 1.8 to about 2, or about 2 to about 2.2. In further embodiments, the microgranules have a width diameter of about 1.6 to about 1.8 mm. According to the disclosure, the microgranules have a height diameter of about 2 to about 3 mm. In some embodiments, the microgranules have a height diameter of about 2, about 2.1, about 2.2, about 2.3, about 2.4, about 2.5, about 2.6, about 2.7, about 2.8, about 2.9, or about 3 mm. In other embodiments, the microgranules have a height diameter of about 2 to about 2.8, about 2 to about 2.6, about 2 to about 2.4, about 2 to about 2.2, about 2.2 to about 3, about 2.2 to about 2.8, about 2.2 to about 2.6, about 2.2 to about 2.4, about 2.4 to about 3, about 2.4 to about 2.8 about 2.4 to about 2.6, about 2.6 to about 3, about 2.6 to about 2.8, or about 2.8 to about 3 mm.

According to the disclosure, the microgranules comprise an extruded core. The term “extruded core” as used herein refers to a core that has been extruded using techniques known in the art. The extruded core is substantially wax-free. The term “substantially wax-free” as used herein refers to the core comprising less than about 1 wt. % of a wax. In some embodiments, the extruded core contains less than about 0.9, about 0.8, about 0.7, about 0.6, about 0.5, about 0.4, about 0.3, about 0.2, or about 0.1 wt. % of a wax. In other embodiments, the extruded core contains 0 wt. % of a wax. In further embodiments, the extruded core contains about 0 to about 0.8, about 0 to about 0.6, about 0 to about 0.4, about 0 to about 0.2, about 0.2 to about 0.8, about 0.2 to about 0.6, about 0.2 to about 0.4, about 0.4 to about 0.8, about 0.4 to about 0.6, about 0.6 to about 0.8 wt. % of a wax.

The extruded cores of the disclosure and their coatings are substantially free of a wax. Waxes are known in the art and include organic compounds that are lipophilic and solid at or near ambient temperature. The “wax” referenced herein is a vegetal wax, beeswax, paraffin wax, or synthetic wax. In some embodiments, the wax is a vegetal wax, such as carnauba wax, rice bran wax, microcrystalline wax, or a fat. In other embodiments, the wax is carnauba wax. In further embodiments, wax is rice bran wax. In yet other embodiments, the wax is microcrystalline wax. In still further embodiments, the wax is a fat. In other embodiments, the wax is paraffin wax or synthetic wax. In further embodiments, the wax is a paraffin wax. In still other embodiments, the wax is a synthetic wax, such as an edible synthetic wax vegetal wax. In yet further embodiments the wax is beeswax.

According to the disclosure, the extruded core contains a choline salt. In some embodiments, the choline salt is choline chloride, choline bitartrate, citrate dehydrogenated choline, choline bicarbonate, choline sulfate, or choline hydroxide. In other embodiments, the choline salt is choline chloride. In further embodiments, the choline salt is choline bitartrate. In yet other embodiments, the choline salt is citrate dehydrogenated choline. In still further embodiments, the choline salt is choline bicarbonate. In other embodiments, the choline salt is choline sulfate. In further embodiments, the choline salt is choline hydroxide.

According to the disclosure, the extruded core contains about 55 to about 65 wt. % of the coated microgranule, of the choline salt. In some embodiments, the extruded core contains about 55, about 56, about 57, about 58, about 59, about 60, about 61, about 62, about 63, about 64, or about 65 wt. % of the coated microgranule, of the choline salt. In other embodiments, the extruded core contains about 55 to about 64, about 55 to about 63, about 55 to about 62, about 55 to about 61, about 55 to about 60, about 55 to about 59, about 55 to about 58, about 55 to about 57, about 55 to about 56, about 56 to about 65, about 56 to about 64, about 56 to about 63, about 56 to about 62, about 56 to about 61, about 56 to about 60, about 56 to about 59, about 56 to about 58, about 56 to about 57, about 57 to about 65, about 57 to about 64, about 57 to about 63, about 57 to about 62, about 57 to about 61, about 57 to about 60, about 57 to about 59, about 57 to about 58, about 58 to about 65, about 58 to about 64, about 58 to about 63, about 58 to about 62, about 58 to about 61, about 58 to about 60, about 58 to about 59, about 59 to about 65, about 59 to about 64, about 59 to about 63, about 59 to about 62, about 59 to about 61, about 59 to about 60, about 60 to about 65, about 60 to about 64, about 60 to about 63, about 60 to about 62, about 60 to about 61, about 61 to about 65, about 61 to about 64, about 61 to about 63, about 61 to about 62, about 62 to about 65, about 62 to about 64, about 62 to about 63, about 63 to about 65, about 63 to about 64, or about 64 to about 64 wt. % of the coated microgranule, of the choline salt.

The extruded cores of the disclosure can include one or more excipients. Examples of excipients include, for example, magnesium stearate, stearic acid, calcium stearate, lecithin (e.g., sunflower lecithin, rapeseed lecithin, coconut lecithin, soy lecithin, egg yolk lecithin), sodium stearyl fumarate, polyethylene glycols, silicon dioxide, talc, and combinations thereof. In some aspects, the extruded cores include magnesium stearate, talc, and polyethylene glycols as excipients. In some aspects, the extruded cores include stearic acid, talc, and polyethylene glycols as excipients. In some aspects, the extruded cores include calcium stearate, talc, and polyethylene glycols as excipients. In some aspects, the extruded cores include calcium stearate, silicon dioxide, and polyethylene glycols as excipients. In some aspects, the extruded cores include magnesium stearate, silicon dioxide, and polyethylene glycols as excipients. In some aspects, the extruded cores include stearic acid, silicon dioxide, and polyethylene glycols as excipients. In some aspects, the extruded cores include calcium stearate, silicon dioxide, and lecithin as excipients. In some aspects, the extruded cores include magnesium stearate, silicon dioxide, and lecithin as excipients. In some aspects, the extruded cores include stearic acid, silicon dioxide, and lecithin as excipients.

According to the disclosure, the extruded core contains talc. In some embodiments, the extruded core contains about 2.8 to about 3.8 wt. % of the coated microgranule, of talc. In other embodiments, the extruded core contains about 2.8, about 2.9, about 3, about 3.1, about 3.2, about 3.3, about 3.4, about 3.5, about 3.6, about 3.7 or about 3.8 wt. % of the coated microgranule, of talc. In further embodiments, the extruded core contains about 2.8 to about 3.7, about 2.8 to about 3.6, about 2.8 to about 3.5, about 2.8 to about 3.4, about 2.8 to about 3.3, about 2.8 to about 3.1, about 2.8 to about 3, about 2.8 to about 2.9, about 2.9 to about 3.8, about 2.9 to about 3.7, about 2.9 to about 3.6, about 2.9 to about 3.5, about 2.9 to about 3.4, about 2.9 to about 3.3, about 2.9 to about 3.2, about 2.9 to about 3.1, about 2.9 to about 3, about 3 to about 3.8, about 3 to about 3.7, about 3 to about 3.6, about 3 to about 3.5, about 3 to about 3.4, about 3 to about 3.3, about 3 to about 3.2, about 3 to about 3.1, about 3.1 to about 3.8, about 3.1 to about 3.8, about 3.1 to about 3.7, about 3.1 to about 3.6, about 3.1 to about 3.5, about 3.1 to about 3.4, about 3.1 to about 3.3, about 3.1 to about 3.2, about 3.2 to about 3.8, about 3.2 to about 3.7, about 3.2 to about 3.6, about 3.2 to about 3.5, about 3.2 to about 3.4, about 3.2 to about 3.3, about 3.3 to about 3.8, about 3.3 to about 3.7, about 3.3 to about 3.6, about 3.3 to about 3.5, about 3.3 to about 3.4, about 3.4 to about 3.8, about 3.4 to about 3.7, about 3.4 to about 3.6, about 3.4 to about 3.5, about 3.5 to about 3.8, about 3.5 to about 3.7, about 3.5 to about 3.6, about 3.6 to about 3.8, about 3.7 to about 3.7, or about 3.7 or about 3.8 wt. % of the coated microgranule, of talc.

In some embodiments, the extruded core contains about 1.4 to about 2 wt. % of the coated microgranule, of talc. In other embodiments, the extruded core contains about 1.4, about 1.5, about 1.6, about 1.7, about 1.8, about 1.9, or about 2 wt. % of the coated microgranule, of talc. In further embodiments, the extruded core contains about 1.4 to about 1.9, about 1.4 to about 1.8, about 1.4 to about 1.7, about 1.4 to about 1.6, about 1.4 to about 1.5, about 1.5 to about 2, about 1.5 to about 1.9, about 1.5 to about 1.8, about 1.5 to about 1.7, about 1.5 to about 1.6, about 1.6 to about 2, about 1.6 to about 1.9, about 1.6 to about 1.8, about 1.6 to about 1.7, about 1.7 to about 2, about 1.7 to about 1.9, about 1.7 to about 1.8, about 1.8 to about 2, about 1.8 to about 1.9, or about 1.9 to about 2 wt. % of the coated microgranule, of talc.

According to the disclosure, the extruded core contains silicon dioxide. In some embodiments, the extruded core contains about 2.8 to about 3.8 wt. % of the coated microgranule, of silicon dioxide. In other embodiments, the extruded core contains about 2.8, about 2.9, about 3, about 3.1, about 3.2, about 3.3, about 3.4, about 3.5, about 3.6, about 3.7 or about 3.8 wt. % of the coated microgranule, of silicon dioxide. In further embodiments, the extruded core contains about 2.8 to about 3.7, about 2.8 to about 3.6, about 2.8 to about 3.5, about 2.8 to about 3.4, about 2.8 to about 3.3, about 2.8 to about 3.1, about 2.8 to about 3, about 2.8 to about 2.9, about 2.9 to about 3.8, about 2.9 to about 3.7, about 2.9 to about 3.6, about 2.9 to about 3.5, about 2.9 to about 3.4, about 2.9 to about 3.3, about 2.9 to about 3.2, about 2.9 to about 3.1, about 2.9 to about 3, about 3 to about 3.8, about 3 to about 3.7, about 3 to about 3.6, about 3 to about 3.5, about 3 to about 3.4, about 3 to about 3.3, about 3 to about 3.2, about 3 to about 3.1, about 3.1 to about 3.8, about 3.1 to about 3.8, about 3.1 to about 3.7, about 3.1 to about 3.6, about 3.1 to about 3.5, about 3.1 to about 3.4, about 3.1 to about 3.3, about 3.1 to about 3.2, about 3.2 to about 3.8, about 3.2 to about 3.7, about 3.2 to about 3.6, about 3.2 to about 3.5, about 3.2 to about 3.4, about 3.2 to about 3.3, about 3.3 to about 3.8, about 3.3 to about 3.7, about 3.3 to about 3.6, about 3.3 to about 3.5, about 3.3 to about 3.4, about 3.4 to about 3.8, about 3.4 to about 3.7, about 3.4 to about 3.6, about 3.4 to about 3.5, about 3.5 to about 3.8, about 3.5 to about 3.7, about 3.5 to about 3.6, about 3.6 to about 3.8, about 3.7 to about 3.7, or about 3.7 or about 3.8 wt. % of the coated microgranule, of silicon dioxide.

In some embodiments, the extruded core contains about 1.4 to about 2 wt. % of the coated microgranule, of silicon dioxide. In other embodiments, the extruded core contains about 1.4, about 1.5, about 1.6, about 1.7, about 1.8, about 1.9, or about 2 wt. % of the coated microgranule, of silicon dioxide. In further embodiments, the extruded core contains about 1.4 to about 1.9, about 1.4 to about 1.8, about 1.4 to about 1.7, about 1.4 to about 1.6, about 1.4 to about 1.5, about 1.5 to about 2, about 1.5 to about 1.9, about 1.5 to about 1.8, about 1.5 to about 1.7, about 1.5 to about 1.6, about 1.6 to about 2, about 1.6 to about 1.9, about 1.6 to about 1.8, about 1.6 to about 1.7, about 1.7 to about 2, about 1.7 to about 1.9, about 1.7 to about 1.8, about 1.8 to about 2, about 1.8 to about 1.9, or about 1.9 to about 2 wt. % of the coated microgranule, of silicon dioxide.

According to the disclosure, the extruded core contains stearic acid. In some embodiments, the extruded core contains about 1.4 to about 2 wt. % of the coated microgranule, of stearic acid. In other embodiments, the extruded core contains about 1.4, about 1.5, about 1.6, about 1.7, about 1.8, about 1.9, or about 2 wt. % of the coated microgranule, of stearic acid. In further embodiments, the extruded core contains about 1.4 to about 1.9, about 1.4 to about 1.8, about 1.4 to about 1.7, about 1.4 to about 1.6, about 1.4 to about 1.5, about 1.5 to about 2, about 1.5 to about 1.9, about 1.5 to about 1.8, about 1.5 to about 1.7, about 1.5 to about 1.6, about 1.6 to about 2, about 1.6 to about 1.9, about 1.6 to about 1.8, about 1.6 to about 1.7, about 1.7 to about 2, about 1.7 to about 1.9, about 1.7 to about 1.8, about 1.8 to about 2, about 1.8 to about 1.9, or about 1.9 to about 2 wt. % of the coated microgranule, of stearic acid.

According to the disclosure, the extruded core contains sodium stearyl fumarate. In some embodiments, the extruded core contains about 1.4 to about 2 wt. % of the coated microgranule, of sodium stearyl fumarate. In other embodiments, the extruded core contains about 1.4, about 1.5, about 1.6, about 1.7, about 1.8, about 1.9, or about 2 wt. % of the coated microgranule, of sodium stearyl fumarate. In further embodiments, the extruded core contains about 1.4 to about 1.9, about 1.4 to about 1.8, about 1.4 to about 1.7, about 1.4 to about 1.6, about 1.4 to about 1.5, about 1.5 to about 2, about 1.5 to about 1.9, about 1.5 to about 1.8, about 1.5 to about 1.7, about 1.5 to about 1.6, about 1.6 to about 2, about 1.6 to about 1.9, about 1.6 to about 1.8, about 1.6 to about 1.7, about 1.7 to about 2, about 1.7 to about 1.9, about 1.7 to about 1.8, about 1.8 to about 2, about 1.8 to about 1.9, or about 1.9 to about 2 wt. % of the coated microgranule, of sodium stearyl fumarate.

According to the disclosure, the extruded core contains a polyethylene glycol. In some embodiments, the extruded core contains about 1.4 to about 2 wt. % of the coated microgranule, of a polyethylene glycol. In other embodiments, the extruded core contains about 1.4, about 1.5, about 1.6, about 1.7, about 1.8, about 1.9, or about 2 wt. % of the coated microgranule, of a polyethylene glycol. In further embodiments, the extruded core contains about 1.4 to about 1.9, about 1.4 to about 1.8, about 1.4 to about 1.7, about 1.4 to about 1.6, about 1.4 to about 1.5, about 1.5 to about 2, about 1.5 to about 1.9, about 1.5 to about 1.8, about 1.5 to about 1.7, about 1.5 to about 1.6, about 1.6 to about 2, about 1.6 to about 1.9, about 1.6 to about 1.8, about 1.6 to about 1.7, about 1.7 to about 2, about 1.7 to about 1.9, about 1.7 to about 1.8, about 1.8 to about 2, about 1.8 to about 1.9, or about 1.9 to about 2 wt. % of the coated microgranule, of a polyethylene glycol.

According to the disclosure, the extruded core contains magnesium stearate. In some embodiments, the extruded core contains about 1.4 to about 2 wt. % of the coated microgranule, of magnesium stearate. In other embodiments, the extruded core contains about 1.4, about 1.5, about 1.6, about 1.7, about 1.8, about 1.9, or about 2 wt. % of the coated microgranule, of magnesium stearate. In further embodiments, the extruded core contains about 1.4 to about 1.9, about 1.4 to about 1.8, about 1.4 to about 1.7, about 1.4 to about 1.6, about 1.4 to about 1.5, about 1.5 to about 2, about 1.5 to about 1.9, about 1.5 to about 1.8, about 1.5 to about 1.7, about 1.5 to about 1.6, about 1.6 to about 2, about 1.6 to about 1.9, about 1.6 to about 1.8, about 1.6 to about 1.7, about 1.7 to about 2, about 1.7 to about 1.9, about 1.7 to about 1.8, about 1.8 to about 2, about 1.8 to about 1.9, or about 1.9 to about 2 wt. % of the coated microgranule, of magnesium stearate.

According to the disclosure, the extruded core contains calcium stearate. In some embodiments, the extruded core contains about 1.4 to about 2 wt. % of the coated microgranule, of calcium stearate. In other embodiments, the extruded core contains about 1.4, about 1.5, about 1.6, about 1.7, about 1.8, about 1.9, or about 2 wt. % of the coated microgranule, of calcium stearate. In further embodiments, the extruded core contains about 1.4 to about 1.9, about 1.4 to about 1.8, about 1.4 to about 1.7, about 1.4 to about 1.6, about 1.4 to about 1.5, about 1.5 to about 2, about 1.5 to about 1.9, about 1.5 to about 1.8, about 1.5 to about 1.7, about 1.5 to about 1.6, about 1.6 to about 2, about 1.6 to about 1.9, about 1.6 to about 1.8, about 1.6 to about 1.7, about 1.7 to about 2, about 1.7 to about 1.9, about 1.7 to about 1.8, about 1.8 to about 2, about 1.8 to about 1.9, or about 1.9 to about 2 wt. % of the coated microgranule, of calcium stearate.

According to the disclosure, the extruded core contains lecithin. In some embodiments, the extruded core contains about 1.1 to about 2.1 wt. % of the coated microgranule, of lecithin. In other embodiments, the extruded core contains about 1.1, about 1.2, about 1.3, about 1.4, about 1.5, about 1.6, about 1.7, about 1.8, about 1.9, about 2, or about 2.1 wt. % of the coated microgranule, of lecithin. In further embodiments, the extruded core contains about 1.1 to about 2, about 1.1 to about 1.9, about 1.1 to about 1.8, about 1.1 to about 1.7, about 1.1 to about 1.6, about 1.1 to about 1.5, about 1.1 to about 1.4, about 1.1 to about 1.3, about 1.1 to about 1.2, about 1.2 toa about 2.1, about 1.2 to about 2, about 1.2 to about 1.9, about 1.2 to about 1.8, about 1.2 to about 1.7, about 1.2 to about 1.6, about 1.2 to about 1.5, about 1.2 to about 1.4, about 1.2 to about 1.3, about 1.3 to about 2.1, about 1.3 to about 2, about 1.3 to about 1.9, about 1.3 to about 1.8, about 1.3 to about 1.7, about 1.3 to about 1.6, about 1.3 to about 1.5, about 1.3 to about 1.4, about 1.4 to about 2.1, about 1.4 to about 2, about 1.4 to about 1.9, about 1.4 to about 1.8, about 1.4 to about 1.7, about 1.4 to about 1.6, about 1.4 to about 1.5, about 1.5 to about 2.1, about 1.5 to about 2, about 1.5 to about 1.9, about 1.5 to about 1.8, about 1.5 to about 1.7, about 1.5 to about 1.6, about 1.6 to about 2.1, about 1.6 to about 2, about 1.6 to about 1.9, about 1.6 to about 1.8, about 1.6 to about 1.7, about 1.7 to about 2.1, about 1.7 to about 2, about 1.7 to about 1.9, about 1.7 to about 1.8, about 1.8 to about 2.1, about 1.8 to about 2, about 1.8 to about 1.9, about 1.9 to about 2.1, about 1.9 to about 2, or about 2 to about 2.1 wt. % of the coated microgranule, of lecithin. The lecithin included in the extruded core may be a solid or liquid. In some embodiments, the lecithin is a solid such as a powder. In other embodiments, the lecithin is a liquid. The lecithin desirably is a vegetal lecithin. In some embodiments, the lecithin is soy lecithin or sunflower lecithin, or a combination thereof. In other embodiments, the lecithin is soy lecithin. In further embodiments, the lecithin is sunflower lecithin. In yet other embodiments, the lecithin is soy lecithin and sunflower lecithin.

According to the disclosure, the extruded core contains a hydrogenated seed oil, a hydrogenated vegetable oil, or a combination thereof. In some embodiments, the extruded core contains a hydrogenated seed oil. In other embodiments, the extruded core contains a hydrogenated vegetable oil. In further embodiments, the extruded core contains a hydrogenated seed oil and hydrogenated vegetable oil. According to the disclosure, the extruded core contains about 4 to about 5 wt. % of the coated microgranule, of a hydrogenated seed oil, a hydrogenated vegetable oil, or a combination thereof. In some embodiments, the extruded core contains about 4, about 4.1, about 4.2, about 4.3, about 4.4, about 4.5, about 4.6, about 4.7, about 4.8, about 4.9, or about 5 wt. % of the coated microgranule, of a hydrogenated seed oil, a hydrogenated vegetable oil, or a combination thereof. In other embodiments, the extruded core contains about 4 to about 4.9, about 4 to about 4.8, about 4 to about 4.7, about 4 to about 4.6, about 4 to about 4.5, about 4 to about 4.4, about 4 to about 4.3, about 4 to about 4.2, about 4 to about 4.1, about 4.1 to about 5, about 4.1 to about 4.9, about 4.1 to about 4.8, about 4.1 to about 4.7, about 4.1 to about 4.6, about 4.1 to about 4.5, about 4.1 to about 4.4, about 4.1 to about 4.3, about 4.1 to about 4.2, about 4.2 to about 5, about 4.2 to about 4.9, about 4.2 to about 4.8, about 4.2 to about 4.7, about 4.2 to about 4.6, about 4.2 to about 4.5, about 4.2 to about 4.4, about 4.2 to about 4.3, about 4.3 to about 5, about 4.3 to about 4.9, about 4.3 to about 4.8, about 4.3 to about 4.7, about 4.3 to about 4.6, about 4.3 to about 4.5, about 4.3 to about 4.4, about 4.4 to about 5, about 4.4 to about 4.9, about 4.4 to about 4.8, about 4.4 to about 4.7, about 4.4 to about 4.6, about 4.4 to about 4.5, about 4.5 to about 5, about 4.5 to about 4.9, about 4.5 to about 4.8, about 4.5 to about 4.7, about 4.5 to about 4.6, about 4.6 to about 5, about 4.6 to about 4.9, about 4.6 to about 4.8, about 4.6 to about 4.7, about 4.7 to about 5, about 4.7 to about 4.9, about 4.7 to about 4.8, about 4.8 to about 5, about 4.8 to about 4.9, or about 4.9 to about 5 wt. % of the coated microgranule, of a hydrogenated seed oil, a hydrogenated vegetable oil, or a combination thereof. In further embodiments, the extruded core contains about 4.1 to about 4.8 wt. % of the coated microgranule, of a hydrogenated seed oil, a hydrogenated vegetable oil, or a combination thereof. In yet other embodiments, the extruded core contains about 4.2 to about 4.3 wt. % of the coated microgranule, of a hydrogenated seed oil, a hydrogenated vegetable oil, or a combination thereof. The hydrogenated vegetable oil in the extruded core may have a melting point of about 60 to about 75° C. In some embodiments, the hydrogenated vegetable oil in the extruded core has a melting point of about 60, about 65, about 70, or about 75° C. In other embodiments, the hydrogenated vegetable oil in the extruded core has a melting point of about 65 to about 75, about 65 to about 70, or about 70 to about 75° C. The hydrogenated vegetable oil in the extruded core also may have a degree of hydrogenation of about 95% or greater. The degree of hydrogenation of a hydrogenated vegetable oil can be determined by methods known to those skilled in the art. For example, the NGD C32-1976 method may be used to determine the degree of hydrogenation. In some embodiments, the degree of hydrogenation is expressed in terms of “iodine value.” In some embodiments, the hydrogenated vegetable oil in the extruded core also may have a degree of hydrogenation of about 95, about 96, about 97, about 98, about 99, or about 100%.

The hydrogenated vegetable oil in the extruded core also may have free fatty acid content of about 2 wt. % or less, by weight of the hydrogenated vegetable oil. In some embodiments, hydrogenated vegetable oil in the extruded core also may have free fatty acid content of about 2 wt. % or less, about 1.5 wt. % or less, about 1 wt. % or less, or about 0.5 wt. % or less, or about 0.1 wt. % or less. One skilled in the art would be able to determine the “free fatty acid” content. For example, the “NGD C10-1976 method” may be used to determine fatty acid content. In some embodiments, the fatty acid is palmitic acid or stearic acid.

In certain embodiments, the hydrogenated vegetable oil is hydrogenated soybean oil, hydrogenated cottonseed oil, hydrogenated sunflower seed oil, or hydrogenated palm oil, or a combination thereof. In some embodiments, the hydrogenated vegetable oil is hydrogenated soybean oil. In other embodiments, the hydrogenated vegetable oil is hydrogenated cottonseed oil. In further embodiments, the hydrogenated vegetable oil hydrogenated palm oil. The hydrogenated seed oil in the extruded core may be hydrogenated rapeseed (canola) oil or hydrogenated linseed oil. In some embodiments, the hydrogenated seed oil is hydrogenated rapeseed (canola) oil. In other embodiments, the hydrogenated seed oil is hydrogenated linseed oil. In further embodiments, the hydrogenated seed oil is hydrogenated sunflower seed oil.

According to the disclosure, the microgranules include one or more substantially wax free coatings. The substantially wax free coating is provided over the extruded core. In some embodiments, the microgranules contain one substantially free wax free coating. Desirably, the coating comprises less than about 1 wt. % of a wax. The term “substantially wax-free” as used herein refers to the coating comprising less than about 1 wt. % of a wax. In some embodiments, the coating contains less than about 0.9, about 0.8, about 0.7, about 0.6, about 0.5, about 0.4, about 0.3, about 0.2, or about 0.1 wt. % of a wax. In other embodiments, the coating contains 0 wt. % of a wax. In further embodiments, the extruded core contains about 0 to about 0.8, about 0 to about 0.6, about 0 to about 0.4, about 0 to about 0.2, about 0.2 to about 0.8, about 0.2 to about 0.6, about 0.2 to about 0.4, about 0.4 to about 0.8, about 0.4 to about 0.6, about 0.6 to about 0.8 wt. % of a wax, based on the weight of the coated microgranule. In further embodiments, the extruded core contains about 0 to about 0.8, about 0 to about 0.6, about 0 to about 0.4, about 0 to about 0.2, about 0.2 to about 0.8, about 0.2 to about 0.6, about 0.2 to about 0.4, about 0.4 to about 0.8, about 0.4 to about 0.6, about 0.6 to about 0.8 wt. % of a wax, based on the weight of the coating.

The coating also does not include an amount of palmitic acid beyond a naturally occurring amount. In some embodiments, the coating comprises less than about 1 wt. % of palmitic acid, based on the weight of the coating. In some embodiments, the coating contains less than about 0.9, about 0.8, about 0.7, about 0.6, about 0.5, about 0.4, about 0.3, about 0.2, or about 0.1 wt. % of palmitic acid. In other embodiments, the coating contains 0 wt. % of palmitic acid. In further embodiments, the extruded core contains about 0 to about 0.8, about 0 to about 0.6, about 0 to about 0.4, about 0 to about 0.2, about 0.2 to about 0.8, about 0.2 to about 0.6, about 0.2 to about 0.4, about 0.4 to about 0.8, about 0.4 to about 0.6, about 0.6 to about 0.8 wt. % of palmitic acid, based on the weight of the coating.

According to the disclosure, the substantially wax free coating contains a hydrogenated seed oil, palm oil, or a combination thereof. In some embodiments, the substantially wax free coating contains hydrogenated seed oil. In other embodiments, the substantially wax free coating contains palm oil. In further embodiments, the substantially wax free coating contains hydrogenated seed oil and palm oil. The substantially wax free coating contains about 26 to about 32 wt. % of the coated microgranule of a hydrogenated seed oil, palm oil, or a combination thereof, based on the weight of the coated microgranule. In some embodiments, the substantially wax free coating contains about 26, about 27, about 28, about 29, about 30, about 31, or about 32 wt. % of the coated microgranule of a hydrogenated seed oil, palm oil, or a combination thereof. In other embodiments, the substantially wax free coating contains about 26 to about 31, about 26 to about 30, about 26 to about 29, about 26 to about 28, about 26 to about 27, about 27 to about 32, about 27 to about 31, about 27 to about 30, about 27 to about 29, about 27 to about 28, about 28 to about 32, about 28 to about 31, about 28 to about 30, about 28 to about 29, about 29 to about 32, about 29 to about 31, about 29 to about 30, about 30 to about 32, about 30 to about 31, or about 31 to about 32 wt. % of the coated microgranule (based on the weight of the coated microgranule) of a hydrogenated seed oil, palm oil, or a combination thereof.

According to the disclosure, the hydrogenated seed oil in the coating is hydrogenated soybean oil, hydrogenated rapeseed (canola) oil, hydrogenated cottonseed oil, hydrogenated palm oil, or hydrogenated linseed oil. In some embodiments, the hydrogenated seed oil in the coating is hydrogenated soybean oil. In other embodiments, the hydrogenated seed oil in the coating is hydrogenated rapeseed (canola) oil. In further embodiments, the hydrogenated seed oil in the coating is hydrogenated cottonseed oil. In yet other embodiments, the hydrogenated seed oil in the coating is hydrogenated palm oil. In still further embodiments, the hydrogenated seed oil in the coating is hydrogenated linseed oil.

According to the disclosure, the coated microgranules contain about 65 to about 75 wt. % of the extruded core, based on the weight of the coated microgranule. In some embodiments, the coated microgranules contain about 65, about 66, about 67, about 68, about 69, about 70, about 71, about 72, about 73, about 74, or about 75 wt. % of the extruded core, based on the weight of the coated microgranule. In other embodiments, the coated microgranules contain about 65 to about 74, about 65 to about 73, about 65 to about 72, about 65 to about 71, about 65 to about 70, about 65 to about 69, about 65 to about 68, about 65 to about 67, about 65 to about 66, about 66 to about 75, about 66 to about 74, about 66 to about 73, about 66 to about 72, about 66 to about 71, about 66 to about 70, about 66 to about 69, about 66 to about 68, about 66 to about 67, about 67 to about 75, about 67 to about 74, about 67 to about 73, about 67 to about 72, about 67 to about 71, about 67 to about 70, about 67 to about 69, about 67 to about 68, about 68 to about 75, about 68 to about 74, about 68 to about 73, about 68 to about 72, about 68 to about 71, about 68 to about 70, about 68 to about 69, about 69 to about 75, about 69 to about 74, about 69 to about 73, about 69 to about 72, about 69 to about 71, about 69 to about 70, about 70 to about 75, about 70 to about 74, about 70 to about 73, about 70 to about 72, about 70 to about 71, about 71 to about 75, about 71 to about 74, about 71 to about 73, about 71 to about 72, about 72 to about 75, about 72 to about 74, about 72 to about 73, about 73 to about 75, about 73 to about 74, or about 74 to about 75 wt. % of the extruded core, based on the weight of the coated microgranule.

According to the disclosure, the coated microgranules contain about 25 to about 35 wt. % of the coating, based on the weight of the coated microgranule. In some embodiments, the coated microgranules contain about 25, about 26, about 27, about 28, about 29, about 30, about 31, about 32, about 33, about 34, or about 35 wt. % of the coating, based on the weight of the coated microgranule. In other embodiments, the coated microgranules contain about 25 to about 34, about 25 to about 33, about 25 to about 32, about 25 to about 31, about 25 to about 30, about 25 to about 29, about 25 to about 28, about 25 to about 27, about 25 to about 26, about 26 to about 35, about 26 to about 34, about 26 to about 33, about 26 to about 32, about 26 to about 31, about 26 to about 30, about 26 to about 29, about 26 to about 28, about 26 to about 27, about 27 to about 35, about 27 to about 34, about 27 to about 33, about 27 to about 32, about 27 to about 31, about 27 to about 30, about 27 to about 29, about 27 to about 28, about 28 to about 35, about 28 to about 34, about 28 to about 33, about 28 to about 32, about 28 to about 31, about 28 to about 30, about 28 to about 29, about 29 to about 35, about 29 to about 34, about 29 to about 33, about 29 to about 32, about 29 to about 31, about 29 to about 30, about 30 to about 35, about 30 to about 34, about 30 to about 33, about 30 to about 32, about 30 to about 31, about 31 to about 35, about 31 to about 34, about 31 to about 33, about 31 to about 32, about 32 to about 35, about 32 to about 34, about 32 to about 33, about 33 to about 35, about 33 to about 34, or about 34 to about 35 wt. % of the coating, based on the weight of the coated microgranule.

The microgranules may contain optional components, as determined by one skilled in the art. In some embodiments, the optional component is included in the core. In other embodiments, the optional component is included in the coating. The optional component may be selected from one or more of methionine, lysine, lysine hydrochloride, lysine sulfate, vitamins (e.g., vitamin C, vitamin C derivative, vitamin A, vitamin A derivative, vitamin E, vitamin E derivative, vitamin D3, vitamin D3 derivative, vitamin B1, vitamin B1 derivative, vitamin B2, vitamin B2 derivative, vitamin B6, vitamin B6 derivative, vitamin B12, vitamin H, vitamin PP, vitamin PP derivative), acids (e.g., lactic acid, fumaric acid, sorbic acid, formic acid, citric acid, malic acid, acetic acid, butyric acid, or propionic acid), silicates (e.g., colloidal silica, synthetic amorphous silica, precipitated silica, sodium aluminum silicates, calcium silicate, kaolin, hydrophobic synthetic zeolites), amino acids, enzymes, nutrients (e.g., proteins and carbohydrates), probiotic microorganisms, prebiotic foods, mineral salts, fatty acids (e.g., lauric acid, oleic acid, linoleic acid, linolenic acid,), emulsifiers (e.g., ethoxylated castor oil, sodium alginate, magnesium alginate), disintegrants (e.g., carboxymethyl cellulose (CMC), dry inserted corn starch, ethoxylated castor oil, gum arabic, or polymethyl methacrylate (PMMA)), or hydrophobic substances, esters of fatty acids, esters of fatty alcohols, stearin, sodium glyceryl mono-stearate, sodium glyceryl di-stearate, sodium glyceryl tri-stearate, stearyl alcohol, cetylstearyl alcohol).

The microgranules may be prepared using techniques and steps known in the art. For example, processes for preparing the microgranules may include one or more steps of extrusion (e.g., bi-screw or twin extruder), heating, cooling (e.g., spray-cooling), cutting (e.g., cutter with variable speed, strand cutting), sieving, spheronization (e.g., a rotating disk spheronizer), compression, dripping, coacervation, or pelletization.

### Feedstuff/Methods of Use

The microgranules described herein have a wide uses for veterinary use. In some embodiments, feedstuff are provided and contain the coated microgranules described herein. The feedstuff may be administered to a ruminant animal, as needed in the art. The term “ruminant animal” as used herein refers to a hoofed mammal having a digestive system intended for plant-only diets and a stomach with a rumen as the first compartment.

The feedstuff may contain optional components, as selected by one skilled in the art. In some embodiments, the optional feedstuff components include, without limitation, grasses (e.g., oat straw, barley straw, wheat straw), legumes, and other fibrous plant material. In other embodiments, the optional feedstuff components are native or cultivated.

It is an advantage of the microgranules described herein that they minimize delivery of choline to the rumen of the ruminant animal. Instead, the microgranules permit delivery of the choline to the omasum, abomasum, or a combination thereof of the ruminant animal. In some embodiments, choline is delivered to the omasum of the ruminant animal. In other embodiments, choline is delivered to the abomasum of the ruminant animal. In further embodiments, choline is delivered to the omasum and abomasum of the ruminant animal. Doing so enhances and optimizes bioavailability of choline for the ruminant animal.

Thus, the disclosure provides methods of delivering choline to the lower digestive tract to a ruminant animal. The methods include administering the feedstuff described herein to the ruminant animal. By doing so, the choline delivered by the microgranules is biologically available. The term “biologically available” as used herein refers to at least some of the choline in the coated microgranules of the disclosure is absorbed by the ruminant animal for use as a nutrient.

The degree of post-ruminal digestibility of the microgranules may be determined using techniques known in the art including, e.g., the Boisen method. For a description of the Boisen method, reference is made to Boisen, “Prediction of total tract digestibility of energy feedstuffs and pig diets by in vitro analyses,” Animal Feed Science Technology 68, 277-286, 1997 and WO-2018/154532, which are incorporated by reference herein. In some embodiments, the degree of post-ruminal digestibility may be determined using the following Boisen test parameters:
39° C., 30 rpm, 2 h, buffer solution with “gastric” pH (2.0)+pepsin inoculum.
39° C., 30 rpm, 4 h, buffer solution with “intestinal” pH (6.8)+pancreatine inoculum.
39° C., 30 rpm, 18 h, buffer solution with “intestinal” pH (6.8)+lipase and bile extract inoculum.

The degree of ruminal bypass may be determined using techniques known in the art. For example, the degree of ruminal bypass is determined at 39° C., 15 rpm, 8 hours presence in the buffer aqueous solution with “ruminal” pH of about 6.8.

Desirably, after administration of the coated microgranules to the ruminant animal, the bioavailability of the choline administered is high, as measured by those skilled in the art. One particular method of determining bioavailability is the Boisen method as described above. In some embodiments, the bioavailability of the choline administered is at least about 70%. In some embodiments, the bioavailability of the choline administered is at least about 70, about 75, about 80, about 85, about 90, about 95, or about 99%. In other embodiments, the bioavailability of the choline administered is 100%.

### EXAMPLES

The following examples, while illustrative individual embodiments, are not intended to limit the scope of the described invention, and the reader should not interpret them in this way.

### Example 1

Microgranules of the invention, including a core and a coating, were prepared Each microgranule included about 60-80 wt. % of the core and about 20-40 wt. % of the coating (based on the weight of the microgranule). The core includes about 55% to about 65 wt. % of choline chloride and about 2 to about 6 wt. % of hydrogenated rapeseed (canola) oil, plus additional excipients. The coating includes a mixture of hydrogenated rapeseed (canola) oil and palm oil.

All ingredients fed in powder form (solid state) in the extruder. Choline chloride with a purity of 99% was mixed with a spray congealed mixture of hydrogenated rapeseed (canola) oil and excipients. The mixture was extruded using an extruder with 10 sectors set at 70-80° C. The cores thus obtained had a concentration of 84-85% in choline chloride.

The cores were subsequently subjected to coating in a pan coater. The coating layer was formed, coating the microgranules with the coating mixture comprising: hydrogenated rapeseed (canola) oil and palm oil.

All the above indicated percentages are percentages by weight based on the total weight of the covering layer. The coated microgranules thus obtained had a concentration of 60% to about 80% by weight of choline chloride with respect to the total weight of the coated microgranules.

### Example 2

Microgranules of the invention, including a core and a coating, were prepared Each microgranule included about 60-80 wt. % of the core and about 20-40 wt. % of the coating (based on the weight of the microgranule). The core includes about 55% to about 65 wt. % of choline chloride and about 2 to about 6 wt. % of hydrogenated soybean oil, plus additional excipients. The coating includes a mixture of hydrogenated soybean oil and palm oil.

All ingredients fed in powder form (solid state) in the extruder. Choline chloride with a purity of 99% was mixed with a spray congealed mixture of hydrogenated soybean oil and excipients. The mixture was extruded using an extruder with 10 sectors set at 70-80° C. The cores thus obtained had a concentration of 84-85% in choline chloride.

The cores were subsequently subjected to coating in a pan coater. The coating layer was formed, coating the microgranules with the coating mixture comprising: hydrogenated soybean oil and palm oil.

All the above indicated percentages are percentages by weight based on the total weight of the covering layer. The coated microgranules thus obtained had a concentration of 60% to about 80% by weight of choline chloride with respect to the total weight of the coated microgranules.

### Example 3: Bioavailability Studies

### A. 2-Layer Prior Art Microgranules (Pan Coater)

Two-layer prior art microgranules containing the components noted in Table 2 were prepared.

TABLE 2

| | Component | Wt. % | |
|---|---|---|---|
| Core (71.5%) | Choline chloride | 60.47 | |
| | crystals | | |
| | Rice bran wax | 4.03 | |
| | Soy lecithin | 2.02 | |
| | Calcium stearate | 1.42 | |
| | Silica | 3.56 | |
| Coating | Hydrogenated | 15.44 | Layer 1 |
| | rapeseed | | |
| | (canola) oil | | |
| (28.5%) | Palmitic Acid | 2.85 | |
| | Distilled | 0.71 | |
| | Monoglyceride | | |
| | Hydrogenated | 4.75 | Layer 2 |
| | rapeseed | | |
| | (canola) oil | | |
| | Rice bran wax | 4.75 | |
| | | 100.00 | |

These prior art microgranules were then analyzed. The results showed that over a period of 12 months, bypass (Boisen method, step 1)=(84.3±6.8) %, digestibility (Boisen method, step 2)=(60.5±20.5) %, bioavailability=bypass*digestibility=(50.5±16.4) %. These data are the average of batches having a bypass≥70%).

B. Wax Free, One-Layer with Hydrogenated Rapeseed (Canola) Oil (in Both Core and Coating Layer Applied in a Pan Coater)

Example 1 was run on a pan coater at fixed process conditions and coating layer composition, bypass (Boisen method, step 1)=(86.5±6.3) %, digestibility (Boisen method, step 2)=(90.1±4.1) %, bioavailability=(77.8±4.8) %.

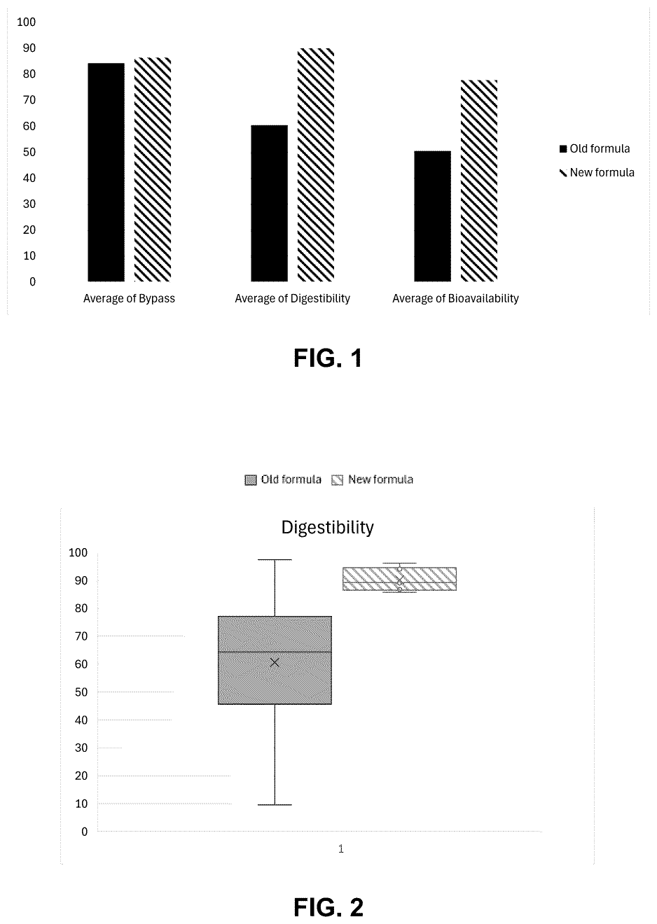

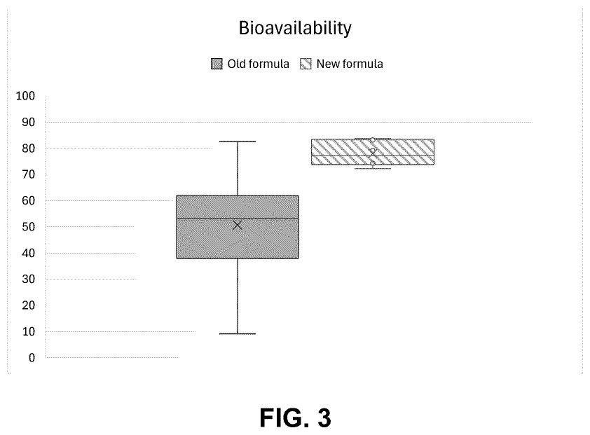

### See, FIGS. 1-3.

### Example 4

Choline chlorine with a purity of 99% were mixed with a spray congealed mixture of rice bran wax and excipients. The mixture was extruded using an extruder with 10 sectors set at temperature range 90-98° C. The cores had a choline chloride concentration of 85% by weight.

The cores were subsequently subjected to coating in a pan. The coating layer was formed by coating the microgranules with a coating mixture comprising 85-93% by weight of hydrogenated soybean oil and 7-15% by weight of palm oil. See, Table 3.

TABLE 3

| | Soy/Palm oil |
|---|---|
| Sample # | weight ratio |
| 1 | 93:7 |
| 2 | 90:10 |
| 3 | 90:10 |
| 4 | 90:10 |
| 5 | 85:15 |
| 6 | 85:15 |

The coated microgranules had a concentration of 60% by weight of choline chloride with respect to the total weight of the microgranules.

The microgranules then were analyzed for degree of ruminal bypass, post-ruminal digestibility and total bioavailability using the Boisen method. See, Table 4.

TABLE 4
Pan coater results

| | Ruminal | | Total |
|---|---|---|---|
| | Bypass | Digestibility | Bioavailability |
| Sample # | (%) | (%) | (%) |
| 1 | 92 | 82 | 75 |
| 2 | 91 | 95 | 87 |
| 3 | 95 | 89 | 85 |
| 4 | 93 | 93 | 86 |
| 5 | 72 | 96 | 69 |
| 6 | 63 | 97 | 62 |

The results showed that, for batches showing bypass greater than 60%, digestibility was greater than about 80%.

What is claimed is:

1. A coated microgranule, comprising:
(a) an extruded core that is substantially wax-free and comprises:
about 55 to about 65 wt. % of the coated microgranule, of a choline salt;
about 4 to about 5 wt. % of the coated microgranule, of a hydrogenated seed oil or a hydrogenated vegetable oil;
one or more excipients; and
(b) a substantially wax free coating comprising about 26 to about 32 wt. % of the coated granule of a hydrogenated seed oil, palm oil, or a combination thereof.

2. The coated microgranule of claim 1, wherein the coating does not include an amount of palmitic acid beyond a naturally occurring amount.

3. The coated microgranule of claim 1, wherein the coating comprises less than about 1 wt. % of palmitic acid.

4. The coated microgranule of claim 1, wherein the coating (b) comprises less than about 1 wt. % of a wax.

5. The coated microgranule of claim 1, wherein core (a) comprises less than about 1 wt. % of a wax.

6. The coated microgranule of claim 1, wherein the choline salt is choline chloride, choline bitartrate, citrate dehydrogenated choline, choline bicarbonate, choline sulfate, or choline hydroxide, preferably choline chloride.

7. The coated microgranule of claim 1, comprising about 55 to about 64, or about 55 to about 63, or about 55 to about 62, or about 55 to about 61, or about 55 to about 60, or about 55 to about 59, or about 55 to about 58, or about 55 to about 57, or about 55 to about 56, or about 56 to about 65, or about 56 to about 64, or about 56 to about 63, or about 56 to about 62, or about 56 to about 61, or about 56 to about 60, or about 56 to about 59, or about 56 to about 58, or about 56 to about 57, or about 57 to about 65, or about 57 to about 64, or about 57 to about 63, or about 57 to about 62, or about 57 to about 61, or about 57 to about 60, or about 57 to about 59, or about 57 to about 58, or about 58 to about 65, or about 58 to about 64, or about 58 to about 63, or about 58 to about 62, or about 58 to about 61, or about 58 to about 60, or about 58 to about 59, or about 59 to about 65, or about 59 to about 64, or about 59 to about 63, or about 59 to about 62 wt. %, or about 59 to about 61, or about 59 to about 60, or about 60 to about 65, or about 60 to about 64, or about 60 to about 63, or about 60 to about 62, or about 60 to about 61 wt. %, or about 61 to about 65, or about 61 to about 64, or about 61 to about 63, or about 61 to about 62, or about 62 to about 65, or about 62 to about 64, or about 62 to about 63, or about 63 to about 65, or about 63 to about 64, or about 64 to about 64 wt. % of the coated microgranule, of the choline salt.

8. The coated microgranule of claim 1, comprising about 4 to about 4.9, or about 4 to about 4.8, or about 4 to about 4.7, or about 4 to about 4.6, or about 4 to about 4.5, or about 4 to about 4.4, or about 4 to about 4.3, or about 4 to about 4.2, or about 4 to about 4.1, or about 4.1 to about 5, or about 4.1 to about 4.9, or about 4.1 to about 4.8 wt. %, or about 4.1 to about 4.7, or about 4.1 to about 4.6, or about 4.1 to about 4.5, or about 4.1 to about 4.4, or about 4.1 to about 4.3, or about 4.1 to about 4.2, or about 4.2 to about 5, or about 4.2 to about 4.9, or about 4.2 to about 4.8, or about 4.2 to about 4.7, or about 4.2 to about 4.6, or about 4.2 to about 4.5, or about 4.2 to about 4.4, or about 4.2 to about 4.3, or about 4.3 to about 5, or about 4.3 to about 4.9, or about 4.3 to about 4.8, or about 4.3 to about 4.7, or about 4.3 to about 4.6, or about 4.3 to about 4.5, or about 4.3 to about 4.4, or about 4.4 to about 5, or about 4.4 to about 4.9, or about 4.4 to about 4.8, or about 4.4 to about 4.7, or about 4.4 to about 4.6, or about 4.4 to about 4.5, or about 4.5 to about 5, or about 4.5 to about 4.9, or about 4.5 to about 4.8, or about 4.5 to about 4.7, or about 4.5 to about 4.6, or about 4.6 to about 5, or about 4.6 to about 4.9, or about 4.6 to about 4.8, or about 4.6 to about 4.7, or about 4.7 to about 5, or about 4.7 to about 4.9, or about 4.7 to about 4.8, or about 4.8 to about 5, or about 4.8 to about 4.9, or about 4.9 to about 5 wt. %, of the coated microgranule, of the hydrogenated seed oil or hydrogenated vegetable oil in the core (a).

9. The coated microgranule of claim 1, wherein the hydrogenated seed oil in (a) is hydrogenated soybean oil, hydrogenated rapeseed (canola) oil, hydrogenated cottonseed oil, hydrogenated palm oil, or hydrogenated linseed oil, or such as rapeseed (canola) oil, or such as soybean oil.

10. The coated microgranule of claim 1, wherein the hydrogenated seed oil in (b) is hydrogenated soybean oil, hydrogenated rapeseed (canola) oil, hydrogenated cottonseed oil, hydrogenated palm oil, or hydrogenated linseed oil, or such as rapeseed (canola) oil, or such as soybean oil.

11. The coated microgranule of claim 1, wherein the wax is a vegetal wax, such as carnauba wax, rice wax, microcrystalline wax, or a fat.

12. The coated microgranule of claim 1, wherein the wax is paraffin max or synthetic wax, such as an edible synthetic wax vegetal wax, such as carnauba wax, rice wax, microcrystalline wax, or a fat.

13. The coated microgranule of claim 1, comprising about 65 to about 75, or about 65 to about 74, or about 65 to about 73, or about 65 to about 72, or about 65 to about 71, or about 65 to about 70, or about 65 to about 69, or about 65 to about 68, or about 65 to about 67, or about 65 to about 66, or about 66 to about 75, or about 66 to about 74, or about 66 to about 73, or about 66 to about 72, or about 66 to about 71, or about 66 to about 70, or about 66 to about 69, or about 66 to about 68, or about 66 to about 67, or about 67 to about 75, or about 67 to about 74, or about 67 to about 73, or about 67 to about 72, or about 67 to about 71, or about 67 to about 70, or about 67 to about 69, or about 67 to about 68, or about 68 to about 75, or about 68 to about 74, or about 68 to about 73, or about 68 to about 72, or about 68 to about 71, or about 68 to about 70, or about 68 to about 69, or about 69 to about 75, or about 69 to about 74, or about 69 to about 73, or about 69 to about 72, or about 69 to about 71, or about 69 to about 70, or about 70 to about 75, or about 70 to about 74, or about 70 to about 73, or about 70 to about 72, or about 70 to about 71, or about 71 to about 75, or about 71 to about 74, or about 71 to about 73, or about 71 to about 72, or about 72 to about 75, or about 72 to about 74, or about 72 to about 73, or about 73 to about 75, or about 73 to about 74, or about 74 to about 75 wt. % of the extruded core (a).

14. The coated microgranule of claim 1, comprising about 25 to about 35, or about 25 to about 34, or about 25 to about 33, or about 25 to about 32, or about 25 to about 31, or about 25 to about 30, or about 25 to about 29, or about 25 to about 28, or about 25 to about 27, or about 25 to about 26, or about 26 to about 35, or about 26 to about 34, or about 26 to about 33, or about 26 to about 32, or about 26 to about 31, or about 26 to about 30, or about 26 to about 29, or about 26 to about 28, or about 26 to about 27, or about 27 to about 35, or about 27 to about 34, or about 27 to about 33, or about 27 to about 32, or about 27 to about 31, or about 27 to about 30, or about 27 to about 29, or about 27 to about 28, or about 28 to about 35, or about 28 to about 34, or about 28 to about 33, or about 28 to about 32, or about 28 to about 31, or about 28 to about 30, or about 28 to about 29, or about 29 to about 35, or about 29 to about 34, or about 29 to about 33, or about 29 to about 32, or about 29 to about 31, or about 29 to about 30, or about 30 to about 35, or about 30 to about 34, or about 30 to about 33, or about 30 to about 32, or about 30 to about 31, or about 31 to about 35, or about 31 to about 34, or about 31 to about 33, or about 31 to about 32, or about 32 to about 35, or about 32 to about 34, or about 32 to about 33, or about 33 to about 35, about 33 to about 34, or about 34 to about 35 wt. % of the coating (b).

15. A feedstuff comprising the coated microgranule of claim 1.

16. A method of delivering choline to the lower digestive tract to a ruminant animal, comprising administering the feedstuff of claim 15 to the ruminant animal.

17. The method of claim 16, wherein the delivered choline is biologically available.

18. The method of claim 16, wherein the choline is delivered to the omasum of the ruminant animal.

19. The method of claim 16, wherein the choline is delivered to the abomasum of the ruminant animal.

20. The method of claim 16, wherein the bioavailability of the choline administered to the ruminant animal is at least about 70%.

21. The method of claim 16, wherein the bioavailability of the choline administered to the ruminant animals is determined by the Boisen method.
